(19) United States
(12) Patent Application Publication
(10) Pub. No.: US 2021/0308284 A1
(43) Pub. Date: Oct. 7, 2021

(54) METAL ATOM CLUSTER-EMBEDDED MAGNETIC IRON OXIDE NANOPARTICLE (MION), AND PREPARATION METHOD AND APPLICATION THEREOF

(71) Applicant: XI'AN SUPERMAG BIO-NANOTECH CO., LTD., Xi'an (CN)

(72) Inventors: Haiming FAN, Xi'an (CN); Xiaoli LIU, Xi'an (CN); Mingli PENG, Xi'an (CN)

(73) Assignee: XI'AN SUPERMAG BIO-NANOTECH CO., LTD., Xi'an (CN)

(21) Appl. No.: 17/266,627

(22) PCT Filed: Mar. 18, 2019
(86) PCT No.: PCT/CN2019/078427

§ 371 Date: Feb. 8, 2021

(30) Foreign Application Priority Data
Nov. 7, 2018 (CN) 201811316448.9

(51) Int. Cl. A61K 49/18; C01G 49/02; H01F 1/058

(52) U.S. Cl. CPC A61K 49/1887; B82Y 25/00; H01F 1/058; C01G 49/02

(57) ABSTRACT

A metal atom cluster-embedded magnetic iron oxide nanoparticle (MION) is disclosed. The metal atom cluster is embedded in an iron oxide crystal matrix and has a content of 0.1% to 15%. A method for preparing the MION includes: dissolving a metal precursor of iron oxide, an organic acid, and an organic amine in an organic solvent to form a uniform reaction system; heating the reaction system to 150° C. to 350° C. in an inert gas atmosphere; adding a metal atom cluster precursor; and heating to perform a reflux reaction until the metal atom cluster precursor is completely decomposed. The MION shows improved magnetic properties due to the embedding of the metal atom cluster, and the iron oxide fully ensures the stability of properties of the nanoparticles. The nanoparticles are especially applicable to biomedical detection and therapy and other fields.

### CROSS REFERENCE TO THE RELATED APPLICATIONS

This application is the national phase entry of International Application No. PCT/CN2019/078427, filed on Mar. 18, 2019, which is based upon and claims priority to Chinese Patent Application No. 201811316448.9, filed on Nov. 7, 2018, the entire contents of which are incorporated herein by reference.

### TECHNICAL FIELD

The present invention relates to the technical field of magnetic iron oxide, and in particular, to a metal atom cluster-embedded magnetic iron oxide nanoparticle (MION), and a preparation method and an application thereof.

### BACKGROUND

In recent years, the research of magnetic nanoparticles has attracted widespread interest in various disciplines. Iron oxide nanomaterials, as an important magnetic material, has excellent biocompatibility, which can be widely used in biological separation and detection, targeted drugs, and medical imaging in addition to magnetic fluids, catalysts, and magnetic recording materials. Iron oxide has been preclinically or clinically used in iron supplements (such as ferumoxytol), magnetic resonance imaging (MRI) contrast agents (such as Combidex®), magnetic hyperthermia agents (such as NanoTherm® approved by the European Supervisory Authority), and drug carriers. In order to prepare MIONs with high biocompatibility and excellent and stable magnetic properties, U.S. Pat. No. 6,262,129; Chinese patent Nos. CN200580040484.1, CN200480044382.2 and CN02820174.4; J. Am. Chem. Soc., 1999, 121 (49), 11595 published by the research team of Alivisatos; J. Am. Chem. Soc., 2002, 124, 8204 published by the research team of Sun; J. Am. Chem. Soc., 2004, 126 (1), 273; J. Am. Chem. Soc., 2001, 123 (51), 12798 published by the research team of Hyeon; Nat. Mater., 2004, 3 (12), 891; Peng, X. Chem. Mater., 2004, 16, 393; and other documents all disclose the use of high-temperature pyrolysis to prepare uniform magnetic nanoparticles of ferrite, iron oxide, iron and an alloy thereof, and the like.

Although the MIONs prepared by the above method have uniform sizes and morphologies and stable superparamagnetic properties, these MIONs show low magnetic responsiveness, insufficient imaging sensitivity, low magneto-thermal conversion efficiency, and other problems in MM, cell tracking, and magneto-thermal conversion. Therefore, improving the stability and magnetic properties of magnetic nanomaterials are very much an active area of current research. Common methods to improve the magnetic properties of MIONs include: preparing ferrite nanoparticles with a spinel structure; preparing cubic ferrite nanoparticles; forming ferrite core/shell nanostructures with an exchange coupling effect; and other strategies.

However, the particles prepared by these methods have several shortcomings. Ferrite or cubic morphology, for example, results in limited improvement in magnetic properties and corresponding application performance, and ferrite core/shell nanostructures require preparation processes that are especially complex, which makes a reaction process difficult to control.

### SUMMARY

In order to meet the demand in biomedical applications for MIONs with high saturation magnetization and stable properties, the present invention provides a metal atom cluster-embedded MION and a preparation method and an application thereof.

In order to achieve the above objective, the present invention adopts the following technical solutions.

A metal atom cluster-embedded MION, where the metal atom cluster is embedded in an iron oxide crystal matrix, and the metal atom cluster has a content of 0.1% to 15% in the metal atom cluster-embedded MION.

The metal atom cluster has a particle size of 0.2 nm to 5 nm, and the iron oxide crystal matrix has a particle size of 2 nm to 100 nm.

Preferably, the MION has a particle size of 3 nm to 50 nm.

Preferably, the metal atom cluster may be an Mₓ cluster formed by a metal atom M, with the x ranging from 3 to 100, and the M may be at least one selected from the group consisting of a rare earth metal, a fourth-period transition metal, and a post-transition metal.

More preferably, the M may be at least one selected from the group consisting of Fe, Co, Ni, Mn, Ga, Nd, Sm, Tb, Dy, Ho, Er, Tm, Yb, and Ce.

Due to the interaction between the metal atom cluster and the iron oxide matrix, the metal atom cluster-embedded MION provided in the present invention has prominent biocompatibility, stable properties, and improved saturation magnetization.

In order to achieve the above objective, the present invention also provides a method for preparing the metal atom cluster-embedded MION, including the following steps:

S1: dissolving a metal precursor of iron oxide, an organic acid, and an organic amine in an organic solvent at a predetermined ratio to form a uniform reaction system; and

S2: heating the reaction system obtained in S1 to 150° C. to 350° C. in an inert gas atmosphere; adding a metal atom cluster precursor; and heating to perform a reflux reaction until the precursor is completely decomposed to obtain the metal atom cluster-embedded MION.

The metal precursor may be an iron-containing organic complex and the metal atom cluster precursor may be a metal organic complex. The iron-containing organic complex may include: iron erucate, ferric acetylacetonate (Fe(acac)₃), ferric oleate (Fe(OA)₃), iron pentacarbonyl (Fe(CO)₅), or iron N-nitrosophenylhydroxylamine (FeCup₃); and the metal organic complex may include: ferric acetylacetonate (Fe(acac)₃), ferric oleate (Fe(OA)₃), iron pentacarbonyl (Fe(CO)₅), iron N-nitrosophenylhydroxylamine (FeCup₃), Co₂(CO)₈, Co(acac)₂, Ni(OOCCH₃)₂, Ni(acac)₂, an oleate-rare earth complex, or an acetylacetonate-rare earth complex.

The organic acid and the organic amine have a molar ratio of 1:(0.5-10); the organic acid and the organic solvent have a volume ratio of 1:(1-100); the organic amine and the organic solvent have a volume ratio of 1:(1-100); and the metal precursor has a concentration of 0.01 mol/L to 1 mol/L.

Preferably, the organic acid may have a carbon chain length of 6 to 25; the organic amine may have a carbon chain length of 6 to 25; and the organic solvent may be a reducing solvent.

More preferably, the organic acid may be one of oleic acid, stearic acid, and erucic acid; the organic amine may be one of oleylamine and octadecylamine (ODA); and the organic solvent may be one of trioctylamine, tributylamine, 1,2-hexadecanediol, and octylamine.

The reaction in S2 may be conducted at 200° C. to 360° C. for 0.5 h to 8 h.

In the method for preparing the metal atom cluster-embedded MION provided in the present invention, based on the high-temperature pyrolysis of a metal precursor, a metal atom cluster embedded in an iron oxide crystal is formed through the reduction or doping of a solvent to obtain metal atom cluster-embedded MIONs, which is simple and controllable.

The present invention also provides an application of the metal atom cluster-embedded MION in fields of MRI, long-term cell tracking, and magnetic nanoparticle imaging.

Advantages

1. The metal atom cluster-embedded MION of the present invention is a magnetic nanoparticle in which a metal atom cluster is embedded in an iron oxide crystal. The MION shows significantly-improved magnetic properties due to the embedding of the metal atom cluster, and the iron oxide matrix fully ensures the stability of properties of the nanoparticles. Therefore, the nanoparticles are especially applicable to biomedical detection and therapy, and other fields.

2. The present invention adopts the metal precursor pyrolysis method, where the size and morphology of nanoparticles can be controlled by controlling reactant concentration, reaction time and reaction temperature.

3. The metal atom cluster-embedded MION of the present invention can be applied to fields of MRI, long-term cell tracking, magnetic nanoparticle imaging, and the like, which have important practical significance.

### BRIEF DESCRIPTION OF THE DRAWINGS

FIG. 1 is a transmission electron microscopy (TEM) image of the elemental iron cluster-embedded MION according to Example 1 of the present invention;

FIG. 2 is a high-resolution transmission electron microscopy (HRTEM) image of the elemental iron cluster-embedded MION according to Example 1 of the present invention;

FIG. 3 is a selected area electron diffraction (SAED) image of the elemental iron cluster-embedded MION according to Example 1 of the present invention;

FIG. 4 is an X-ray diffraction (XRD) pattern of the elemental iron cluster-embedded MION according to Example 1 of the present invention; and

FIG. 5 is a diagram showing a hysteresis loop of the elemental iron cluster-embedded MION according to Example 1 of the present invention.

### DETAILED DESCRIPTION OF THE EMBODIMENTS

The present invention relates to a metal atom cluster-embedded MION, as well as a preparation method and an application thereof. In the present invention, a metal precursor of iron oxide, an organic acid, and an organic amine are dissolved in an organic solvent at a predetermined ratio to form a uniform reaction system; the reaction system is heated to 150° C. to 350° C. in an inert gas atmosphere; a metal atom cluster precursor is added; a resulting mixture is heated and a reflux reaction is carried out until the precursor is completely decomposed to obtain metal atom cluster-embedded MIONs; and finally, the metal atom cluster-embedded MIONs obtained are used in fields of MM, long-term cell tracking, magnetic nanoparticle imaging, and the like.

In the present invention, the metal precursor may be an iron-containing organic complex, including, but not limited to: iron erucate, ferric acetylacetonate (Fe(acac)₃), ferric oleate (Fe(OA)₃), iron pentacarbonyl (Fe(CO)₅), and iron N-nitrosophenylhydroxylamine (FeCup₃).

The metal atom cluster precursor may be a metal organic complex, including: an iron organic complex, specifically ferric acetylacetonate (Fe(acac)₃), ferric oleate (Fe(OA)₃), iron pentacarbonyl (Fe(CO)₅), or iron N-nitrosophenylhydroxylamine (FeCup₃); a cobalt organic complex, specifically Co₂(CO)₈ or Co(acac)₂; a nickel organic complex, specifically Ni(OOCCH₃)₂ or Ni(acac)₂; and a gadolinium organic complex, specifically Gd(OA)₃ or Gd(acac)₃. The metal atom cluster precursor is not limited to the above substances.

The organic acid may have a carbon chain length of 6 to 25, specifically one of oleic acid, stearic acid, and erucic acid; the organic amine may have a carbon chain length of 6 to 25, specifically one of oleylamine and ODA; and the organic solvent may be a reducing solvent, specifically one of trioctylamine, tributylamine, 1,2-hexadecanediol, and octylamine.

The composition of the iron oxide is (Fe₂O₃)ᵣ(Fe₃O₄)₁₋ᵣ, with r ranging from 0 to 1.

The present invention is described in detail below with reference to specific examples.

### Example 1

Preparation method of an iron cluster-embedded MION (iron cluster iron oxide, ICIO): ferric acetylacetonate (Fe(acac)₃, 0.4 mmol), oleic acid (6 mmol), oleylamine (6 mmol), and trioctylamine (30 mL) were thoroughly mixed under stirring in a nitrogen atmosphere to obtain a uniform mixture. The mixture was heated to 200° C. and kept at this temperature for 1 h, and ferric acetylacetonate (Fe(acac)₃, 0.05 mmol) was added at an increased nitrogen flow; a resulting mixture was heated to 340° C. and reacted at reflux for 2 h to obtain a black-brown mixture; and the black-brown mixture was naturally cooled to room temperature. 10 mL of alcohol was added to the black-brown mixture to precipitate a black substance, and a resulting solution was then centrifuged; the black substance obtained by centrifugation was dissolved in 10 mL of n-hexane, and a resulting solution was centrifuged at 5,000 rpm for 10 min to remove undispersed residue; a supernatant obtained by centrifugation was subjected to precipitation with alcohol; and a resulting solution was centrifuged at 5,000 rpm for 10 min to remove the solvent to obtain the iron cluster-embedded MION.

A series of characterizations were conducted on the prepared iron cluster-embedded MION. Specifically, the iron cluster-embedded MION was dispersed in n-hexane, then 2 μL of the solution of nanoparticles in n-hexane was dropped on a carbon film-coated Cu mesh, which was naturally dried for characterizations. FIG. 1 is a TEM image, and it can be seen from FIG. 1 that the iron cluster-embedded MION is uniform in size and morphology, with monodispersity and a size of about 20 nm.

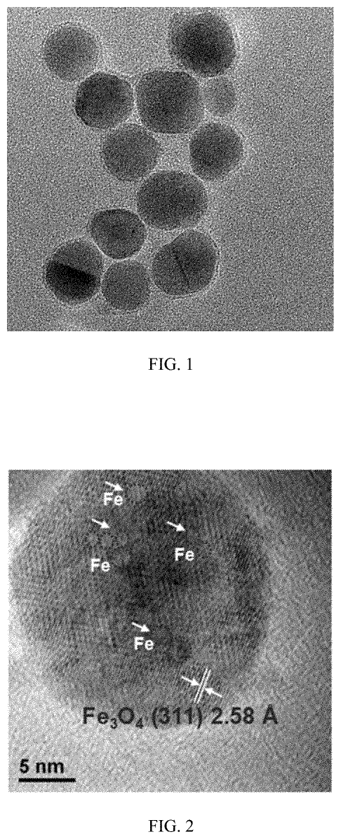

FIG. 2 is an HRTEM image, and it can be seen from FIG. 2 that there are lattice fringes, indicating that the nanoparticles have a high crystallinity; the lattice spacing is 0.258 nm, which is in line with the vertical spacing of the (311) lattice plane, indicating that the nanoparticles are iron oxide nanoparticles; and more importantly, there are Fe clusters embedded in the iron oxide nanoparticle lattices.

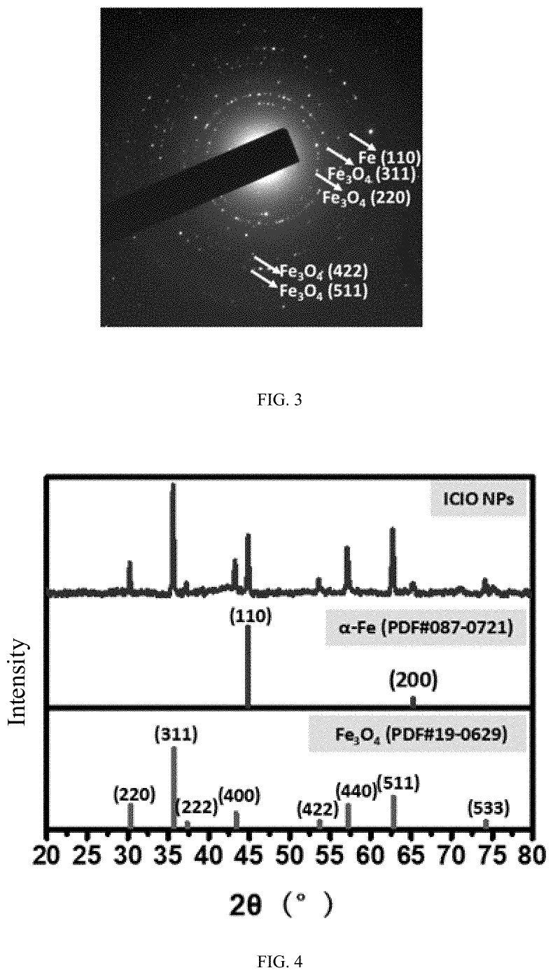

FIG. 3 is an SAED image, and it can be further confirmed from FIG. 3 that there are Fe clusters in iron oxide particles.

FIG. 4 is an XRD pattern, which indicates that the nanoparticles are well crystallized and there are peaks of the Fe phase and peaks of the reverse crystal Fe₃O₄ phase.

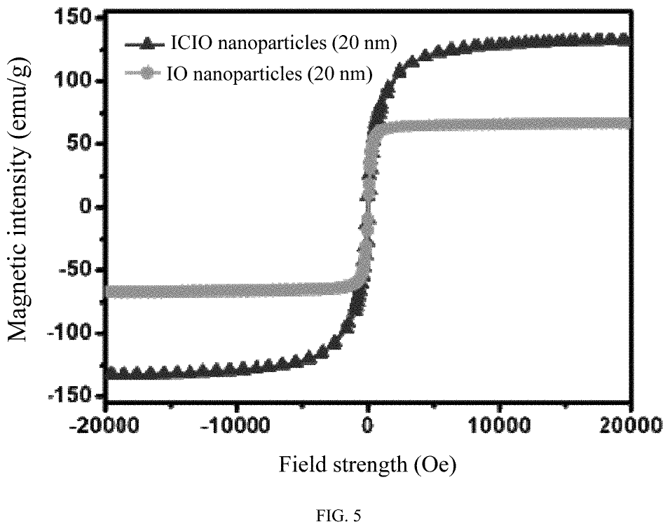

FIG. 5 shows the VSM characterization results, which indicate that the ICIO prepared in this example has high stability due to the embedding of iron clusters in the iron oxide crystals. After being placed for at least one year, the sample still had a measured saturation magnetization value as high as 120 emu/g, but the iron oxide particles without iron clusters prepared under the same conditions had a saturation magnetization value of only 60 emu/g. It further indicates that the iron cluster-embedded MIONs prepared by the method of the present invention have an extremely-high saturation magnetization value and stable properties, and thus can be stored for a long time.

### Example 2

Preparation method of an iron cluster-embedded MION (iron cluster iron oxide, ICIO): ferric oleate (Fe(OA)₃, 0.4 mmol), erucic acid (8 mmol), ODA (4 mmol), and octylamine (40 mL) were thoroughly mixed under stirring in a nitrogen atmosphere to obtain a uniform mixture. The mixture was heated to 150° C. and kept at this temperature for 1 h, and ferric acetylacetonate (Fe(acac)₃, 0.05 mmol) was added at an increased nitrogen flow; a resulting mixture was heated to 200° C. and reacted at reflux for 8 h to obtain a black-brown mixture; and the black-brown mixture was naturally cooled to room temperature. The subsequent treating process was the same as that in Example 1.

### Example 3

Preparation method of an iron cluster-embedded MION (iron cluster iron oxide, ICIO): iron pentacarbonyl (Fe(CO)₅, 0.04 mmol), stearic acid (1 mmol), oleylamine (10 mmol), and tributylamine (40 mL) were thoroughly mixed under stirring in a nitrogen atmosphere to obtain a uniform mixture. The mixture was heated to 300° C. and kept at this temperature for 1 h, and ferric oleate (Fe(OA)₃, 0.005 mmol) was added at an increased nitrogen flow; a resulting mixture was heated to 360° C. and reacted at reflux for 0.5 h to obtain a black-brown mixture; and the black-brown mixture was naturally cooled to room temperature. The subsequent treating process was the same as that in Example 1.

### Example 4

Preparation method of a cobalt cluster-embedded MION (cobalt cluster iron oxide, CCIO): ferric acetylacetonate (Fe(acac)₃, 8 mmol), oleic acid (6 mmol), oleylamine (6 mmol), and trioctylamine (30 mL) were thoroughly mixed under stirring in a nitrogen atmosphere to obtain a uniform mixture. The mixture was heated to 200° C. and kept at this temperature for 1 h, and cobalt carbonyl (Co₂(CO)₈, 1 mmol) was added at an increased nitrogen flow; a resulting mixture was heated to 340° C. and reacted at reflux for 2 h to obtain a black-brown mixture; and the black-brown mixture was naturally cooled to room temperature for subsequent treating. The subsequent treating process was the same as that in Example 1. After the subsequent treating, the cobalt cluster-embedded MIONs were obtained.

### Example 5

Preparation method of a nickel cluster-embedded MION (nickel cluster iron oxide, NCIO): ferric acetylacetonate (Fe(acac)₃, 8 mmol), oleic acid (6 mmol), oleylamine (6 mmol), and trioctylamine (30 mL) were thoroughly mixed under stirring in a nitrogen atmosphere to obtain a uniform mixture. The mixture was heated to 200° C. and kept at this temperature for 1 h, and nickel acetylacetonate (Ni(acac)₂, 1 mmol) was added at an increased nitrogen flow; a resulting mixture was heated to 340° C. and reacted at reflux for 2 h to obtain a black-brown mixture; and the black-brown mixture was naturally cooled to room temperature for subsequent treating. The subsequent treating process was the same as that in Example 1. After the subsequent treating, the nickel cluster-embedded MIONs were obtained.

### Example 6

Preparation method of an iron and nickel cluster-embedded MION: ferric acetylacetonate (Fe(acac)₃, 8 mmol), oleic acid (6 mmol), oleylamine (6 mmol), and trioctylamine (30 mL) were thoroughly mixed under stirring in a nitrogen atmosphere to obtain a uniform mixture. The mixture was heated to 200° C. and kept at this temperature for 1 h, and nickel acetylacetonate (Ni(acac)₂, 0.5 mmol) and ferric acetylacetonate (Fe(acac)₃, 0.5 mmol) were added at an increased nitrogen flow; a resulting mixture was heated to 340° C. and reacted at reflux for 2 h to obtain a black-brown mixture; and the black-brown mixture was naturally cooled to room temperature for subsequent treating. The subsequent treating process was the same as that in Example 1. After the subsequent treating, the iron and nickel cluster-embedded MIONs were obtained.

### Example 7

1 mL of a solution of the iron cluster-embedded MION (ICIO, 20 nm) prepared in Example 1 in water (with an iron content of 0.1 mg/mL) and 1 mL of a solution of iron cluster-free MION (SPIO, 20 nm) in water (with an iron content also of 0.1 mg/mL) were taken and added to a 15 mL test tube, separately, and then the test tube was placed in the magnetic coil of a magneto-thermal converter, so that a medium-frequency alternating magnetic field (with a frequency of 488 kHz and a field strength of 600 Oe) was applied to the outside of the test tube. An optical fiber thermocouple probe was used to measure a temperature change, and the specific absorption rate (SAR) of magnetic nanoparticles was determined. The SAR was defined as the thermal energy per unit time that can be generated by a unit mass of iron in an alternating magnetic field, with a unit of Watt/g. The SAR was calculated according to formula (1), and a calculated value could be used for evaluating the magneto-thermal conversion efficiency of magnetic nanoparticles. The magneto-thermal converter used in this example was produced by Shenzhen Shuangping Power Technology Co., Ltd., with a model of SPG-10AB-II. The instrument was also connected to an optical fiber probe to determine the temperature of a sample solution.

Calculation of SAR:

$$SAR = C \frac{Δ T}{Δ t} \frac{1}{{m}_{Fe}} \qquad formula ( 1 )$$

where: C is the specific heat capacity of an aqueous solution (C_{water}=4.18 J/(g.° C.)); ΔT/Δt is the initial slope in a heating curve; and m_{Fe} is the concentration of iron atoms in a magnetic nanoparticle solution. Test results of the magneto-thermal converter in this example showed that the solution of iron cluster-embedded MIONs (ICIO) in water and the solution of iron cluster-free MIONs (SPIO) in water, after undergoing a magnetic field for 30 s, had temperatures increasing from 27.6° C. to 44.2° C. and to 27.8° C., respectively, and the calculated SAR values were 25,600 W/g and 228 W/g, respectively, fully indicating that the magneto-thermal conversion efficiency of the iron cluster-embedded MIONs was much higher than that of the iron cluster-free MIONs at the same concentration.

### Example 8

The iron cluster-embedded MION (ICIO) prepared in example 1 and the iron cluster-free MION (SPION) were dispersed in agarose gel to enable Fe concentrations of 0.01 mM, 0.025 mM, 0.05 mM, 0.1 mM, 0.25 mM, and 0.5 mM separately. 15 mL of each of the samples obtained above was added to a 20 mL glass bottle, and scanning was conducted with a 7 T small animal MRI system (BioSpec 70/20 USR, Bruker, Germany), with agarose gel as a control sample. MRI scanning parameters: TR=2,900 ms, TE=40.06 ms, field of view=35 mm×35 mm, matrix size=256×256, flip angle=90°, and NEX=3. After MM scanning images of the samples were obtained, the Levenberg-Margardt method was used to calculate the relaxation time T₂ values for the samples at different concentrations on the Matlab software, and then the relaxation rate r₂=1/T₂ was calculated. As calculated, the iron cluster-embedded MION (ICIO) and the iron cluster-free MION (SPION) had r₂ values of 1,060 mM⁻¹S⁻¹ and 185 mM⁻¹S⁻¹, respectively, namely, the iron cluster-embedded MION (ICIO) had an r₂ value more than 5 times that of the iron cluster-free MION (SPION), indicating that the iron cluster-embedded MION exhibited imaging performance much higher than that of the iron cluster-free MION.

### Example 9

The iron cluster-embedded MION prepared in Example 1 was used for magnetic nanoparticle imaging by an MPI scanner (Magnetic Insight Inc, MOMENTUM™ Imager), with a frequency of 45 KHz and a magnetic gradient strength of 5.7 T/m. Data were processed by the VivoQuant software. At a concentration of 0.5 mg/mL, the sample had a measured signal intensity reaching 1,169, while iron cluster-free MION only had a signal intensity of 192. The iron cluster-embedded MIONs had a signal intensity 6 times that of an ordinary MION contrast agent, indicating superior imaging performance.

It should be noted that those of ordinary skill in the art can further make several variations and improvements without departing from the inventive concept of the present invention, but such variations and improvements shall all fall within the protection scope of the present invention.

What is claimed is:

1. A metal atom cluster-embedded magnetic iron oxide nanoparticle (MION), wherein a metal atom cluster of the metal atom cluster-embedded MION is embedded in an iron oxide crystal matrix, and the metal atom cluster has a content of 0.1% to 15% in the metal atom cluster-embedded MION.

2. The metal atom cluster-embedded MION according to claim 1, wherein the metal atom cluster has a particle size of 0.2 nm to 5 nm, and the iron oxide crystal matrix has a particle size of 2 nm to 100 nm.

3. The metal atom cluster-embedded MION according to claim 1, wherein the metal atom cluster is an Mₓ cluster formed by a metal atom M, the x ranges from 3 to 100, and the M is at least one selected from the group consisting of a rare earth metal, a fourth-period transition metal, and a post-transition metal.

4. The metal atom cluster-embedded MION according to claim 3, wherein the M is at least one selected from the group consisting of Fe, Co, Ni, Mn, Ga, Nd, Sm, Tb, Dy, Ho, Er, Tm, Yb, and Ce.

5. A method for preparing the metal atom cluster-embedded MION according to claim 1, comprising the following steps:
S1: dissolving a metal precursor of iron oxide, an organic acid, and an organic amine in an organic solvent at a predetermined ratio to form a uniform reaction system; and
S2: heating the uniform reaction system obtained in S1 to 150° C. to 350° C. in an inert gas atmosphere; adding a metal atom cluster precursor to the uniform reaction system to obtain a mixture; and heating the mixture to perform a reflux reaction until the metal atom cluster precursor is completely decomposed to obtain the metal atom cluster-embedded MION.

6. The method for preparing the metal atom cluster-embedded MION according to claim 5, wherein the metal precursor of iron oxide is an iron-containing organic complex and the metal atom cluster precursor is a metal organic complex; the iron-containing organic complex comprises: iron erucate, ferric acetylacetonate (Fe(acac)₃), ferric oleate (Fe(OA)₃), iron pentacarbonyl (Fe(CO)₅), or iron N-nitrosophenylhydroxylamine (FeCup₃); and the metal organic complex comprises: ferric acetylacetonate (Fe(acac)₃), ferric oleate (Fe(OA)₃), iron pentacarbonyl (Fe(CO)₅), iron N-nitrosophenylhydroxylamine (FeCup₃), Co₂(CO)₈, Co(acac)₂, Ni(OOCCH₃)₂, Ni(acac)₂, an oleate-rare earth complex, or an acetylacetonate-rare earth complex.

7. The method for preparing the metal atom cluster-embedded MION according to claim 5, wherein the organic acid and the organic amine have a molar ratio of 1:(0.5-10); the organic acid and the organic solvent have a volume ratio of 1:(1-100); the organic amine and the organic solvent have a volume ratio of 1:(1-100); and the metal precursor of iron oxide has a concentration of 0.01 mol/L to 1 mol/L.

8. The method for preparing the metal atom cluster-embedded MION according to claim 7, wherein the organic acid has a carbon chain length of 6 to 25; the organic amine has a carbon chain length of 6 to 25; and the organic solvent is a reducing solvent.

9. The method for preparing the metal atom cluster-embedded MION according to claim 5, wherein the reflux reaction in S2 is conducted at 200° C. to 360° C. for 0.5 h to 8 h.

10. A method of using the metal atom cluster-embedded MION according to claim 1, comprising using the metal atom cluster-embedded MION in fields of magnetic resonance imaging (MRI), long-term cell tracking, and magnetic nanoparticle imaging.

11. The method for preparing the metal atom cluster-embedded MION according to claim 5, wherein the metal atom cluster has a particle size of 0.2 nm to 5 nm, and the iron oxide crystal matrix has a particle size of 2 nm to 100 nm.

12. The method for preparing the metal atom cluster-embedded MION according to claim 5, wherein the metal atom cluster is an Mₓ cluster formed by a metal atom M, the x ranges from 3 to 100, and the M is at least one selected from the group consisting of a rare earth metal, a fourth-period transition metal, and a post-transition metal.

13. The method for preparing the metal atom cluster-embedded MION according to claim 12, wherein the M is at least one selected from the group consisting of Fe, Co, Ni, Mn, Ga, Nd, Sm, Tb, Dy, Ho, Er, Tm, Yb, and Ce.

14. The method of using the metal atom cluster-embedded MION according to claim 10, wherein the metal atom cluster has a particle size of 0.2 nm to 5 nm, and the iron oxide crystal matrix has a particle size of 2 nm to 100 nm.

15. The method of using the metal atom cluster-embedded MION according to claim 10, wherein the metal atom cluster is an Mₓ cluster formed by a metal atom M, the x ranges from 3 to 100, and the M is at least one selected from the group consisting of a rare earth metal, a fourth-period transition metal, and a post-transition metal.

16. The method of using the metal atom cluster-embedded MION according to claim 15, wherein the M is at least one selected from the group consisting of Fe, Co, Ni, Mn, Ga, Nd, Sm, Tb, Dy, Ho, Er, Tm, Yb, and Ce.
